(19) United States
(12) Patent Application Publication
(10) Pub. No.: US 2026/0216693 A1
(43) Pub. Date: Jul. 30, 2026

(54) METHOD FOR PRODUCING METAL ORGANIC FRAMEWORK

(71) Applicant: TOAGOSEI CO., LTD., Tokyo (JP)

(72) Inventors: Mayu HANAI HAYAKAWA, Aichi (JP); Yoshinao YAMADA, Aichi (JP)

(73) Assignee: TOAGOSEI CO., LTD., Tokyo (JP)

(21) Appl. No.: 19/144,234

(22) PCT Filed: Dec. 12, 2023
(86) PCT No.: PCT/JP2023/044476

§ 371 Date: Jun. 27, 2025

(30) Foreign Application Priority Data
Dec. 28, 2022 (JP) 2022-212323

(51) Int. Cl. B01J 20/22; B01J 20/28; B01J 20/30; C07F 7/00

(52) U.S. Cl. CPC B01J 20/226; B01J 20/28059; B01J 20/3085; C07F 7/003

(57) ABSTRACT

A method for producing a metal organic framework, the method including: a step of preparing an aqueous zirconium complex solution containing a zirconium atom, and a step of sequentially or simultaneously mixing the aqueous zirconium complex solution with an organic ligand and an alcohol, to obtain a metal organic framework

### TECHNICAL FIELD

The present invention relates to a method for producing a metal organic framework.

### BACKGROUND ART

Metal organic frameworks are porous three-dimensional structures formed from central metals and organic ligands. They are also called MOFs and have been widely studied in recent years. MOFs (hereinafter, also referred to as “MOFs”) are characterized by high specific surface area and freedom of design. In addition, many of them have uniform micropores, so they are used as adsorbents and storage agents for gases.

A conventional method for producing an MOF is a solvothermal synthesis method. Specifically, a metal ion, an organic ligand, and a solvent are put into a reaction vessel and heated to produce an MOF.

Solvothermal methods generally require high temperature reaction, exceeding 100° C., to promote the reaction.

Non-Patent Document 1 describes a method for producing UiO-66, which is a zirconium metal organic framework.

### PRIOR ART DOCUMENT

### Non-Patent Document

Non-Patent Document 1: Cryst. Growth Des., 2020, 20, 10, 6787-6795

### SUMMARY OF INVENTION

### Technical Problem

Water is a versatile and inexpensive solvent, but it is easily coordinated to metals and interferes with synthesis reaction of metal organic frameworks, so there have been few examples of its use as a synthesis solvent for metal organic frameworks.

Non-Patent Document 1 describes a synthesis of a zirconium-based metal organic framework using water, but the crystallinity and specific surface area were low. There was also room for improvement in yield.

The problem to be solved by the present invention is to provide a production method that can obtain a metal organic framework with excellent crystallinity in high yield.

### Solution to Problem

Means for solving the problem above include the following aspects.

<1> A method for producing a metal organic framework, the method including:
a step of preparing an aqueous zirconium complex solution containing a zirconium complex, and
a step of sequentially or simultaneously mixing the aqueous zirconium complex solution with an organic ligand and an alcohol, to obtain a metal organic framework.

<2> The method for producing a metal organic framework according to <1>, in which a liquid temperature during mixing in the step of obtaining a metal organic framework is 80° C. or lower.

<3> The method for producing a metal organic framework according to <1> or <2>, further including a step of adjusting a pH of the aqueous zirconium complex solution to from 2 to 9 before the step of obtaining a metal organic framework.

<4> The method for producing a metal organic framework according to any one of <1> to <3>, in which the organic ligand is a polyvalent carboxylic acid or a salt thereof.

<5> The method for producing a metal organic framework according to any one of <1> to <4>, in which the organic ligand is a polyvalent carboxylic acid having a benzene skeleton or a salt thereof.

<6> The method for producing a metal organic framework according to any one of <1> to <5>, in which a molar ratio of zirconium atoms to organic ligand (zirconium atoms:organic ligand) is from 1:0.1 to 1:3.

<7> The method for producing a metal organic framework according to any one of <1> to <6>, in which, in the step of obtaining a metal organic framework, a mass ratio of water to alcohol (water:alcohol) in the solvent after mixing with the alcohol is from 1:0.1 to 1:3.

<8> The method for producing a metal organic framework according to any one of <1> to <7>, in which, in the step of obtaining a metal organic framework, a content of a compound having a nitrogen atom in the solvent after mixing the aqueous zirconium complex solution, the organic ligand, and the alcohol, is 1% by mass or less.

### Advantageous Effects of Invention

According to the present invention, a method for producing a metal organic framework with excellent crystallinity can be provided.

### BRIEF DESCRIPTION OF DRAWINGS

FIG. 1 is a powder X-ray diffraction measurement (PXRD) spectrum of the metal organic framework obtained in Example 1.

FIG. 2 is a powder X-ray diffraction measurement (PXRD) spectrum of the metal organic framework obtained in Example 2.

FIG. 3 is a powder X-ray diffraction measurement (PXRD) spectrum of the metal organic framework obtained in Comparative Example 1.

### MODES FOR CARRYING OUT INVENTION

The following description of the constituent elements may be based on a representative embodiment of the present invention, but the present invention is not limited to such an embodiment. In the present specification, “to” is used to mean that the numerical values described before and after “to” are included as the lower and upper limits, respectively.

In the numerical ranges described stepwise in the present specification, the upper or lower limit described in one numerical range may be replaced with the upper or lower limit of another numerical range described stepwise. In addition, in the numerical ranges described in the present specification, the upper or lower limit of the numerical range may be replaced with a value shown in Examples.

In the present invention, in a case in which multiple substances corresponding to each component are present in a composition, the amount of each component in the composition means the total amount of the multiple substances present in the composition, unless otherwise specified.

In the present invention, the term “step” includes not only independent step, but also steps that cannot be clearly distinguished from other steps, as long as the intended purpose of the step is achieved.

In the present invention, “% by mass” and “% by weight” are synonymous, and “parts by mass” and “parts by weight” are synonymous.

Further, in the present invention, a combination of two or more preferred aspects is a more preferred aspect.

The present invention is explained in detail below.

### (Method for Producing Metal Organic Framework)

The method for producing a metal organic framework according to the present invention includes a step of preparing an aqueous zirconium complex solution containing a zirconium atom, and a step of sequentially or simultaneously mixing the aqueous zirconium complex solution with an organic ligand and an alcohol, to obtain a metal organic framework.

The metal organic framework produced by the method for producing a metal organic framework according to the present invention can be suitably used as a gas adsorbent or storage agent, or a deodorizer.

The metal organic framework can also be used as a raw material for producing gas adsorption products by blending it with fibers, resins, liquids (e.g., water, organic solvents, etc.).

Examples of applications of gas adsorption products include deodorizing liquids, deodorizing fibers, deodorizing resin compositions, deodorizing fabrics, and deodorizing filter media.

As a result of extensive study, the present inventors have found that by adopting the above-mentioned configuration, a composition with excellent crystallinity can be provided.

The mechanism of the excellent effect thereby is not clear, but it is presumed as follows.

It is presumed that by adding an alcohol to form a mixed solvent of water and the alcohol when mixing an aqueous zirconium complex solution with an organic ligand to produce a metal organic framework, the alcohol having hydrogen donating properties promotes the cleavage of the bond between the zirconium complex and the ligand in a metastable metal organic framework and the re-coordination to a stable structure, thereby obtaining a metal organic framework with excellent crystallinity.

### <Step of Preparing Aqueous Zirconium Complex Solution>

The method for producing a metal organic framework according to the present invention includes a step of preparing an aqueous zirconium complex solution.

In the step of preparing an aqueous zirconium complex solution, it is preferable to prepare the zirconium complex from a metal salt or a hydrate of a metal salt containing a zirconium atom.

The metal salt containing a zirconium atom is not particularly limited. Examples thereof include metal halides (including metal oxyhalides) such as metal chlorides, metal bromides, metal iodides, and metal fluorides; metal nitrates; metal sulfates; metal carbonates; metal formates; metal phosphates; organometallic compounds such as metal carboxylates and metal alkoxides, metallocene compounds, metal sulfides; and metal hydroxides.

More specifically, examples thereof include zirconium tetrachloride, zirconium oxychloride or zirconium oxychloride octahydrate, zirconium nitrate or zirconium nitrate dihydrate, zirconium oxynitrate or zirconium oxynitrate dihydrate, zirconium tetrapropoxide, zirconium tetrabutoxide, and zirconocene dichloride (ZrCp₂Cl₂), but are not limited thereto.

Among these, zirconium oxychloride or its hydrate is preferable as the metal salt or the hydrate of the metal salt containing a zirconium atom.

The solvent used in the step of preparing an aqueous zirconium complex solution is not particularly limited as long as it is a solvent containing water. From the viewpoints of complex formation property and crystallinity of the obtained metal organic framework, the solvent preferably contains 30% by mass or more of water, more preferably contains 80% by mass or more of water, still more preferably contains 90% by mass or more of water, and particularly preferably contains 99% by mass to 100% by mass of water.

In the step of preparing an aqueous zirconium complex solution, it is preferable to add an acid from the viewpoint of complex formation property.

The acid may be an inorganic acid or an organic acid. From the viewpoint of complex formation property, an organic acid is preferable, and a monocarboxylic acid compound is more preferable. The monocarboxylic acid compound is not particularly limited. Examples thereof include acetic acid and formic acid.

From the viewpoint of complex formation property, the addition amount of the acid is preferably from 1 molar equivalent to 1,000 molar equivalents, more preferably from 5 molar equivalents to 800 molar equivalents, and particularly preferably from 10 molar equivalents to 500 molar equivalents, with respect to a usage amount of 1 molar equivalent of zirconium atoms.

It is presumed that the acid becomes a salt by performing the step of adjusting a pH of the aqueous zirconium complex solution to from 2 to 9, which will be described later.

The usage amount of the metal salt or the hydrate of the metal salt containing a zirconium atom and the amount of the solvent are not particularly limited, and can be selected as appropriate.

In particular, the usage amount of the metal salt or the hydrate of the metal salt containing a zirconium atom is preferably from 0.5% by mass to 30% by mass, and more preferably from 1% by mass to 20% by mass, with respect to the total mass of the aqueous zirconium complex solution, from the viewpoint of complex formation property.

The reaction temperature in the step of preparing an aqueous zirconium complex solution is not particularly limited. The reaction temperature is preferably 80° C. or lower. The lower limit of the reaction temperature may be set as appropriate. For example, the lower limit of the reaction temperature may be 5° C. or higher, and may be 20° C. or higher to improve reactivity. The upper limit of the reaction temperature may be set as appropriate. For example, the upper limit of the reaction temperature may be 60° C. or lower.

The reaction time in the step of preparing an aqueous zirconium complex solution is not particularly limited. The reaction time is preferably from 0.1 hours to 72 hours, and more preferably from 1 hour to 36 hours.

<Step of Adjusting pH of Aqueous Zirconium Complex Solution to from 2 to 9>

The method for producing a metal organic framework according to the present invention preferably further includes a step of adjusting a pH of the aqueous zirconium complex solution to from 2 to 9 before the step of obtaining a metal organic framework.

In the step of adjusting a pH of the aqueous zirconium complex solution, it is preferable to add a basic compound to adjust the pH.

The basic compound is not particularly limited. Examples thereof include alkali metal salt compounds and alkaline earth metal salt compounds.

Among them, from the viewpoint of complex formation property, alkali metal salt compounds are preferable, alkali metal carbonate compounds are more preferable, and sodium carbonate is particularly preferable.

The addition amount of the basic compound may be appropriately selected depending on the desired pH.

In the step of adjusting a pH of the aqueous zirconium complex solution to from 2 to 9, it is preferable to adjust the pH to from 3 to 8, and more preferably to from 4 to 7, from the viewpoint of complex formation property.

The pH in the present invention is the value at 25° C., and is measured at 25° C. using a pH measuring device (device name: compact pH meter LAQUA twin AS-pH-22, manufactured by Horiba Advanced Techno Co., Ltd.).

The reaction temperature in the step of adjusting the pH of the aqueous zirconium complex solution to from 2 to 9 before the step of obtaining a metal organic framework is not particularly limited. The reaction temperature is preferably 80° C. or lower.

The reaction time in the step of adjusting the pH of the aqueous zirconium complex solution to from 2 to 9 before the step of obtaining a metal organic framework is not particularly limited. The reaction time is preferably from 0.1 hours to 24 hours, and more preferably from 0.1 hours to 3 hours.

### <Step of Obtaining Metal Organic Framework>

In the method for producing a metal organic framework according to the present invention, the alcohol may be added after mixing the aqueous zirconium complex solution with the organic ligand, or the zirconium complex may be added after mixing the organic ligand and the alcohol, or the organic ligand may be added after mixing the zirconium complex and the alcohol.

The organic ligand used is not particularly limited as long as it can form a metal organic framework by coordinate bonding with the zirconium complex. For example, the organic ligand may have multiple functional groups capable of coordinating with metal ions. Examples of functional groups capable of coordinating with metal ions include a carboxy group, a glycidyl group, a carboxylic anhydride group, CS₂H, OH, SH, SO, SO₂, SO₃H, —S—, —SS—, Si(OH)₃, Ge(OH)₃, Sn(OH)₃, Si(SH)₄, Ge(SH)₄, Sn(SH)₄, PO₃H, CH(SH)₂, C(SH)₃, CH(NH₂)₂, C(NH₂)₃, CH(OH)₂, C(OH)₃, CH(CN)₂, C(CN)₃, CH(RSH)₂, C(RSH)₃, CH(RNH₂)₂, C(RNH₂)₃, CH(ROH)₂, and C(ROH)₃. Among them, functional groups capable of forming salts may be in the form of salts.

The organic ligand may be, for example, an aromatic compound, a heteroaromatic compound, or a heterocyclic compound.

Aromatic compounds refer to monocyclic or polycyclic compounds formed of a 5- or 6-membered aromatic hydrocarbon ring, and specific examples thereof include benzene, naphthalene, 1,4-dihydronaphthalene, fluorene, anthracene, phenanthrene, biphenyl, triphenyl, acenaphthylene, acenaphthene, tetrahydronaphthalene, chroman, 2,3-dihydro-1,4-dioxanaphthalene, pyrene, indane, indene, and phenanthrene.

Heteroaromatic compounds refer to monocyclic or polycyclic compounds formed of a 5- or 6-membered aromatic ring containing 1 to 3 heteroatoms selected from O or S. In the case of polycyclic compounds, at least one ring may be a heteroaromatic ring.

Examples of heterocyclic compounds include morpholine, pyrrolidine, piperidine, methylpiperazine, tetrahydrofuran, and dioxane.

Among these, polycarboxylic acids having an ethylene skeleton and polycarboxylic acids having a benzene skeleton are preferred.

Examples of polycarboxylic acids having an ethylene skeleton include fumaric acid and maleic acid, but are not limited thereto.

Examples of polycarboxylic acids having a benzene skeleton include phthalic acid, isophthalic acid, terephthalic acid, 2,6-naphthalenedicarboxylic acid, and 4,4′-biphenyldicarboxylic acid, but are not limited thereto.

Among these, from the viewpoints of complex formation property and crystallinity of the obtained metal organic framework, dicarboxylic acids consisting of a carboxy group and a divalent hydrocarbon group or salts thereof are preferable, dicarboxylic acids consisting of a carboxy group and a divalent aromatic hydrocarbon group or salts thereof are more preferable, and terephthalic acid or salts of terephthalic acid are particularly preferable. Cations forming salts are not particularly limited. Examples thereof include monovalent cations such as alkali metal ions and ammonium ions.

The terms “divalent hydrocarbon group” and “divalent aromatic hydrocarbon group” each refer to a hydrocarbon group and an aromatic hydrocarbon group obtained by removing two functional groups from an organic ligand having two functional groups capable of coordinating with metal ions.

The usage amount of the organic ligand can be adjusted appropriately according to the amount of the zirconium complex. In particular, from the viewpoint of crystallinity of the obtained metal organic framework, the molar ratio of zirconium atoms to organic ligand (zirconium atoms:organic ligand) contained in the aqueous zirconium complex solution is preferably from 1:0.05 to 1:5, more preferably from 1:0.1 to 1:3, and particularly preferably from 1:0.5 to 1:2.

Furthermore, in the step of obtaining a metal organic framework, the organic ligand may be added as a compound as it is, may be added by dispersing it in a liquid, or may be added as a solution containing the organic ligand.

Examples of solvents for use in the solution containing the organic ligand include water and alcohol, and water is preferable.

The alcohol to be used is not particularly limited. Examples thereof include methanol, ethanol, propanol, and isopropanol.

Among them, it is preferable to use ethanol from the viewpoint of crystallinity of the obtained metal organic framework.

The alcohol may be used singly or in combination of two or more.

In the step of obtaining a metal organic framework, the mass ratio of water to alcohol (water:alcohol) in the solvent after mixing with the alcohol is not particularly limited. From the viewpoints of crystallinity, specific surface area, and yield of the obtained metal organic framework, the mass ratio of water to alcohol (water:alcohol) is preferably from 1:0.1 to 1:3, more preferably from 1:0.1 to 1:2, and particularly preferably from 1:0.2 to 1:2.

In addition, in the step of obtaining a metal organic framework, the total content of water and alcohol in the solvent after mixing the alcohol is preferably 50% by mass or more, may be 80% by mass or more, and may be 100% by mass, with respect to the total mass of the solvent, from the viewpoint of crystallinity of the obtained metal organic framework.

In the step of obtaining a metal organic framework, the content of a compound having a nitrogen atom in the solvent after mixing the alcohol is preferably 10% by mass or less, more preferably 5% by mass or less, and particularly preferably 1% by mass or less, from the viewpoints of crystallinity and yield of the obtained metal organic framework. The lower limit is 0% by mass.

Examples of compounds having a nitrogen atom include compounds having a nitrogen-containing functional group such as an amino group, an amide group, and an imide group, such as N,N-dimethylformamide (DMF), N,N-diethylformamide (DEF), N,N-dimethylacetamide (DMAc), ammonia, and triethylamine.

The reaction temperature in the step of obtaining a metal organic framework is preferably 80° C. or lower from the viewpoints of crystallinity, specific surface area, and yield of the obtained metal organic framework. The lower limit of the reaction temperature may be set appropriately. For example, the lower limit of the reaction temperature may be set to 5° C. or higher, and may be 20° C. or higher to improve reactivity. The upper limit of the reaction temperature may be set appropriately. For example, the upper limit of the reaction temperature may be set to 60° C. or lower.

The reaction time in the step of preparing a metal organic framework is not particularly limited. The reaction time is preferably from 0.05 hours to 72 hours, more preferably from 0.1 hours to 24 hours, and particularly preferably from 0.2 hours to 12 hours.

The method for producing a metal organic framework according to the present disclosure may include other steps than those described above.

Examples of other steps may include known steps. Specific examples thereof include a step of separating the obtained metal organic framework, a step of washing the obtained metal organic framework, and a step of drying the obtained metal organic framework.

The step of drying the obtained metal organic framework may include drying by heating or drying under reduced pressure. When drying by heating, the drying temperature is preferably 200° C. or lower, more preferably 150° C. or lower, and still more preferably 130° C. or lower.

Furthermore, from the viewpoint of suppressing coloring when kneading into a resin, the content of nitrogen atoms in the obtained metal organic framework is preferably 5% by mass or less, more preferably 2% by mass or less, particularly preferably 0.5% by mass or less, and most preferably 0.1% by mass or less. The lower limit is 0% by mass. The content of nitrogen atoms can be measured by Dumas method using a MICRO CORDER JM11, manufactured by J Science Lab, Inc.

From the viewpoint of adsorptivity, the specific surface area of the obtained metal organic framework is preferably 500 m²/g or more, more preferably 700 m²/g or more, particularly preferably 900 m²/g or more, and may be 1,000 m²/g or more.

The half-width of the main peak (peak width at ½ the peak height) in the spectrum obtained by measuring the obtained metal organic framework by powder X-ray diffraction measurement (PXRD) is preferably 0.4° or less, more preferably 0.35° or less, and particularly preferably 0.3° or less.

### EXAMPLES

The present invention will be specifically explained below based on Examples. Note that the present invention is not limited to these Examples. In the following, “parts” and “%” mean “parts by mass” and “% by mass”, respectively, unless otherwise specified.

### <Reagents>

The reagents used in Examples are as follows.
Zirconium oxychloride octahydrate (ZrOCl₂·8H₂O, manufactured by Fujifilm Wako Pure Chemical Industries, Ltd.)
Acetic acid (manufactured by Fujifilm Wako Pure Chemical Industries, Ltd.)
Sodium carbonate (manufactured by Fujifilm Wako Pure Chemical Industries, Ltd.)
Terephthalic acid (manufactured by Tokyo Chemical Industry Co., Ltd.)
Sodium hydroxide (manufactured by Fujifilm Wako Pure Chemical Industries, Ltd.)
Ethanol (manufactured by Fujifilm Wako Pure Chemical Industries, Ltd.)
Methanol (manufactured by Fujifilm Wako Pure Chemical Industries, Ltd.)

### Example 1: Synthesis of Metal Organic Framework (UiO-66)

### <Preparation of Aqueous Zirconium Complex Solution>

3.22 g (10 mmol) of ZrOCl₂·8H₂O and 13.1 g of acetic acid were added to 30 g of pure water and stirred at 50° C. for 24 hours. Then, 1.48 g of sodium carbonate was added to the solution and stirred to adjust the pH (25° C.) to 5.0, thereby preparing an aqueous zirconium complex solution.

### <Preparation of Organic Ligand Solution>

1.66 g (10 mmol) of terephthalic acid and 0.8 g (20 mmol) of sodium hydroxide were added to 50 g of pure water and stirred at 80° C. for 30 minutes to prepare an organic ligand solution.

### <Synthesis of Metal Organic Framework>

At room temperature (25° C., the same applies below), 12.0 g of the organic ligand solution was added dropwise to 9.0 g of the aqueous zirconium complex solution while stirring. Then, 42 g of ethanol was added and stirred at 40° C. for 1 hour. After solid-liquid separation by centrifugation, the solid was collected and washed three times with 50 mL of water, and then heated and dried at 80° C. overnight to obtain the product of Example 1 (metal organic framework UiO-66 ([Zr₆(OH)₄O₄(bdc)₆]), white, particulate, 95% yield). Note that “bdc” represents a terephthalate dianion. The molar ratio of zirconium atoms to organic ligand (zirconium atoms:organic ligand) is 1:1.18. Furthermore, the content of a compound having a nitrogen atom in the product of Example 1 was 0%.

### Example 2: Synthesis of Metal Organic Framework (UiO-66)

### <Preparation of Aqueous Zirconium Complex Solution>

An aqueous zirconium complex solution was prepared in the same manner as in Example 1.

### <Synthesis of Metal Organic Framework>

At room temperature (25° C., the same applies below), 12.0 g of the organic ligand solution was added dropwise to 9.0 g of the aqueous zirconium complex solution while stirring. Then, 10.5 g of ethanol was added and stirred at 40° C. for 1 hour. After solid-liquid separation by centrifugation, the solid was collected and washed three times with 50 mL of water, and then heated and dried at 80° C. overnight to obtain the product of Example 2 (metal organic framework UiO-66 ([Zr₆(OH)₄O₄(bdc)₆]), white, particulate, 92% yield). Note that “bdc” represents a terephthalate dianion. The molar ratio of zirconium atoms to organic ligand (zirconium atoms:organic ligand) is 1:1.18. Furthermore, the content of a compound having a nitrogen atom in the product of Example 2 was 0%.

### Comparative Example 1

The product of Comparative Example 1 (metal organic framework UiO-66) was obtained by the same method as in Non-Patent Document 1, which is specifically as follows.

In a polytetrafluoroethylene container, 1.288 g (4 mmol) of ZrOCl₂·8H₂O and 3 mL of acetic acid were added to 12 mL of pure water, heated at 70° C. for 2 hours, and then cooled slowly to room temperature, thereby preparing an aqueous zirconium complex solution.

0.6640 g (4 mmol) of terephthalic acid and 0.32 g (8 mmol) of sodium hydroxide were added to 20 mL of pure water, mixed, and dissolved to prepare an organic ligand solution.

10 mL of the organic ligand solution was added to a 50 mL centrifuge tube, and 20 M (=20 mol/L) aqueous sodium hydroxide solution, 37% by mass of hydrochloric acid, and pure water were used to adjust the pH to 4.9, thereby preparing 13.5 mL of an organic ligand solution.

10 mL of the aqueous zirconium complex solution was vigorously added to the organic ligand solution using a pipette, and then centrifuged at 12,000 rpm (revolutions per minute) for 5 minutes.

The obtained precipitate was dried in an oven at 70° C. for 8 hours. The obtained sediment was washed with 0.01 M hydrochloric acid, 0.001 M aqueous sodium hydroxide solution, dimethyl sulfoxide, and methanol in this order, and methanol was added to perform solvent replacement overnight. After centrifuging once, drying was performed at 70° C. to obtain the product of Comparative Example 1 (metal organic framework UiO-66, Zr₆(OH)₄O₄(bdc)₆, white, particulate, 74% yield).

### <Evaluation>

Each product obtained was evaluated by the following method, and the evaluation results are shown in Table 1.

### —Crystallinity Evaluation: Analysis Method of Powder X-Ray Diffraction Measurement (PXRD) Spectrum—

The PXRD spectra were analyzed using D8ADVANCE, manufactured by Bruker Japan Co., Ltd. The measurement angle was from 3° to 50°.

The half-width of the main peak of the spectrum of the obtained metal organic framework was calculated using software “DIFFRAC.EVA”, manufactured by Bruker Japan Co., Ltd. The narrower the half-width, the better the crystallinity.

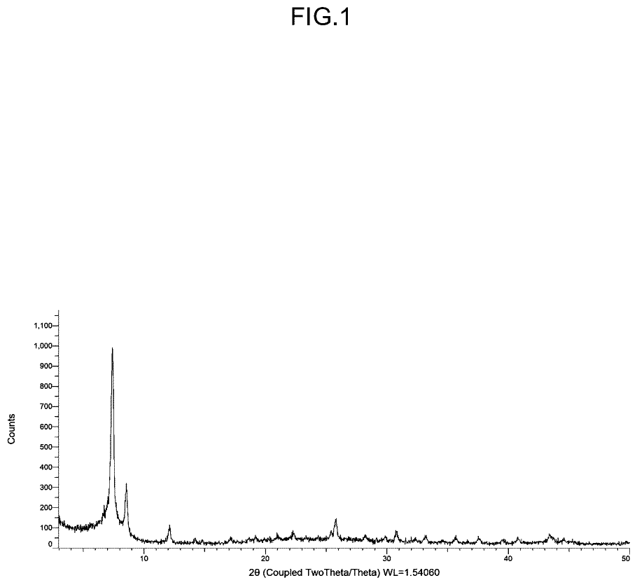

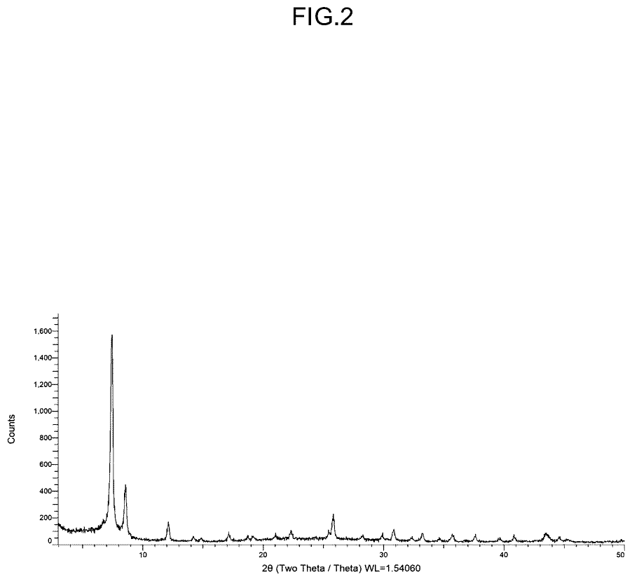

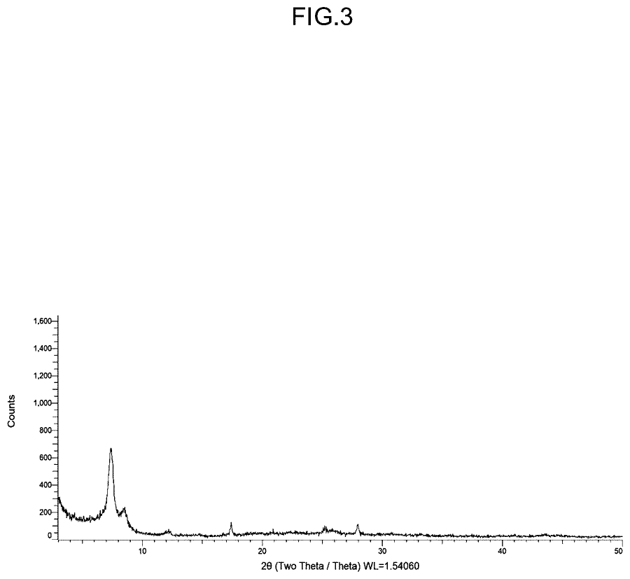

The PXRD (powder x-ray diffraction) spectra obtained by analyzing the metal organic frameworks (UiO-66) produced in Examples 1 and 2 and Comparative Example 1 are shown in FIGS. 1 to 3. FIG. 1 shows the spectrum of the metal organic framework obtained in Example 1, FIG. 2 shows the spectrum of the metal organic framework obtained in Example 2, and FIG. 3 shows the spectrum of the metal organic framework obtained in Comparative Example 1. In each figure, the vertical axis represents a diffraction X-ray intensity (Counts), and the horizontal axis represents a diffraction angle (2θ (°).

### —Measurement of Specific Surface Area—

Using “AUTOSORB-1” manufactured by Anton Paar, the amount of adsorption of nitrogen gas at the boiling point of liquid nitrogen (−195.8° C.) was measured at five points in a relative pressure range of from 0.1 to 0.3, an adsorption isotherm was created, and the BET specific surface area per mass (m²/g) was determined.

TABLE 1

| | Half-width | Specific surface area | Yield |
|---|---|---|---|
| | (°) | (m²/g) | (%) |
| Example 1 | 0.2 | 1.027 | 95 |
| Example 2 | 0.2 | 1.155 | 92 |
| Comparative Example 1 | 0.5 | 455 | 74 |

As shown in Table 1, the metal organic framework obtained by the method for producing a metal organic framework of Examples had better crystallinity than the metal organic framework obtained by the method for producing a metal organic framework of Comparative Example.

Furthermore, as shown in Table 1, the method for producing a metal organic framework of Examples provided a metal organic framework having a large specific surface area, and was also excellent in yield.

The disclosure of Japanese Patent Application No. 2022-212323, filed Dec. 28, 2022, is incorporated herein by reference in its entirety.

All publications, patent applications, and technical standards mentioned in this specification are herein incorporated by reference to the same extent as if each individual publication, patent application, or technical standard was specifically and individually indicated to be incorporated by reference.

1. A method for producing a metal organic framework, the method comprising:
preparing an aqueous zirconium complex solution containing a zirconium complex, and
sequentially or simultaneously mixing the aqueous zirconium complex solution with an organic ligand and an alcohol, to obtain a metal organic framework.

2. The method for producing a metal organic framework according to claim 1, wherein a liquid temperature during mixing in obtaining a metal organic framework is 80° C. or lower.

3. The method for producing a metal organic framework according to claim 1, further comprising adjusting a pH of the aqueous zirconium complex solution to from 2 to 9 before obtaining a metal organic framework.

4. The method for producing a metal organic framework according to claim 1, wherein the organic ligand is a polyvalent carboxylic acid or a salt thereof.

5. The method for producing a metal organic framework according to claim 1, wherein the organic ligand is a polyvalent carboxylic acid having a benzene skeleton or a salt thereof.

6. The method for producing a metal organic framework according to claim 1, wherein a molar ratio of zirconium atoms to organic ligand (zirconium atoms:organic ligand) is from 1:0.1 to 1:3.

7. The method for producing a metal organic framework according to claim 1, wherein, in obtaining a metal organic framework, a mass ratio of water to alcohol (water:alcohol) in the solvent after mixing with the alcohol is from 1:0.1 to 1:3.

8. The method for producing a metal organic framework according to claim 1, wherein, in obtaining a metal organic framework, a content of a compound having a nitrogen atom in the solvent after mixing the aqueous zirconium complex solution, the organic ligand, and the alcohol, is 1% by mass or less.
